(19) United States
(12) Patent Application Publication
(10) Pub. No.: US 2025/0228709 A1
(43) Pub. Date: Jul. 17, 2025

(54) EYE MASK FOR MOISTURE RETENTION

(71) Applicant: Blinkjoy Inc., Encinitas, CA (US)

(72) Inventor: Justin Mendelson, La Jolla, CA (US)

(21) Appl. No.: 19/025,403

(22) Filed: Jan. 16, 2025

Related U.S. Application Data
(60) Provisional application No. 63/621,275, filed on Jan. 16, 2024.
(60) Provisional application No. 63/697,258, filed on Sep. 20, 2024.

(51) Int. Cl. A61F 9/04

(52) U.S. Cl. CPC A61F 9/04

(57) ABSTRACT

An eye mask for treating dry eye and/or irritated eye conditions is provided. The eye mask helps control, maintain and/or increase the temperature and humidity around the eyes to aid in limiting evaporation of natural and/or artificial tears. The eye mask includes two detachable eye covers, each eye cover having a cavity that accommodates the eye or surrounding area. Unlike existing eye masks, the eye mask of the present disclosure may include one or more pressure relief vents to ensure there is no pressure buildup on/around the eye to prevent corneal hypoxia (oxygen deprivation from the cornea).

### CLAIM OF PRIORITY

The present application for patent claims priority to U.S. Provisional Application No. 63/697,258 entitled “Eye Mask for Moisture Retention”, filed Sep. 20, 2024, and U.S. Provisional Application No. 63/621,275 entitled “Sleep Mask for Moisture Retention”, filed Jan. 16, 2024, both of which are hereby expressly incorporated by reference.

### FIELD

The present disclosure relates to eye masks for moisture retention designed to provide relief from symptoms associated with dry eye during sleep.

### BACKGROUND

Dry eye disease (DED) is a common and often uncomfortable condition that affects millions of people worldwide. Tears help maintain the health of the eyes by keeping them moist, nourished and free from irritants. DED, medically known as keratoconjunctivitis sicca, occurs when eyes do not produce sufficient tears (aqueous deficiency) or when tears evaporate too quickly (evaporative dry eye). This can lead to symptoms such as redness, irritation, over tearing, blurred vision, or even painful sensations in the eyes. Most individuals diagnosed with DED primarily experience evaporative dry eye, while a smaller percentage suffer from aqueous deficiency, and an even smaller group is affected by mucin deficiency, neuropathic pain, or other subtypes.

Sleep is an important determinant of health and quality of life. People suffering from DED may experience increased feelings of dryness, pain, or redness throughout the night or after waking up in the morning. In fact, there are many studies that have shown a connection between dry eye and poor sleep quality. Dry eye at night is often caused by both biological and environmental factors, which include:
Decreased Tear Production: Tear production tends to decrease as the day progresses due to the natural circadian rhythm, with the lowest levels occurring at night. This reduction in tear production can leave the eyes more vulnerable to dryness and discomfort.
Decreased Blinking: On average, humans blink about 15-20 times per minute. Blinking is critical for maintaining eye health, responsible for distributing tears evenly across the eye's surface. Unfortunately, in the evening, people tend to read, watch TV, or look at their phone, which decreases our blink rate. This reduction in blinking exacerbates dry eye symptoms as the surface of the eye receives less (and uneven) tear distribution.
Sleeping with Eyes Partially Open: Some individuals sleep with their eyes partially open, a condition known as nocturnal lagophthalmos. This exposes the eyes to more air and increases the rate of tear evaporation, leading to increased dryness.
Corneal Abrasion and Erosion: Corneal erosion occurs when the outer layer of cells on the cornea, known as the epithelium, detaches from the layer beneath. Unfortunately, those with DED are at increased risk for recurrent corneal abrasions, which can in turn increase eye sensitivity and pain, worsening dry eye symptoms.
Medication: Certain medications such as antihistamines can dry out the body and affect tear production or quality.
Environmental Agents: Surrounding airflow from fans and low humidity levels from arid climates or air conditioning can accelerate tear evaporation, leaving the eyes feeling drier and more irritated. Airflow can be particularly problematic for those with nocturnal lagophthalmos (noted above), as eyes are exposed to more air which heightens the risk of evaporative tear loss.

While there are in-office medical treatments available for DED, a simpler at-home therapy to relieve or manage symptoms of dry eye is to sleep with a moisture retaining mask.

A moisture retaining mask is specially designed to create a humid microenvironment around the eyes, helping to maintain a higher level of humidity and prevent excessive evaporation of tears. People who use these devices while sleeping often pair them with nighttime eye drops or nighttime ointments which can replenish or increase the viscosity of tears if a person suffers from low tear production.

Once eyes are properly lubricated, it is important to keep them this way throughout the night, as well hydrated and lubricated eyes expel bacteria and debris more efficiently. Preventing moisture from evaporating also enhances lubrication and reduces sensations of grittiness or irritation.

These devices also serve as a barrier against environmental agents such as wind, dust, pollen, air conditioning, or dry indoor heating that can cause symptom flare ups due to increased tear evaporation or poor tear quality.

Currently, there are no moisture chamber goggles for dry eye relief that are customizable to fit different face sizes/shapes. Furthermore, existing moisture chamber goggles are hard to clean and uncomfortable.

In view of the above, what is needed is an eye mask that can be easily disassembled to clean and has supportive foam padding to increase comfort while sleeping. Furthermore, an eye mask that is designed for side sleeping and includes a mechanism to prevent buildup on/around the eye as well as prevent corneal hypoxia (oxygen deprivation from the cornea) is needed.

### SUMMARY

The following presents a simplified summary of one or more implementations in order to provide a basic understanding of some implementations. This summary is not an extensive overview of all contemplated implementations and is intended to neither identify key or critical elements of all implementations nor delineate the scope of any or all implementations. Its sole purpose is to present some concepts of one or more implementations in a simplified form as a prelude to the more detailed description that is presented later.

According to one example, an eye cover is provided. The eye cover includes an outer silicone layer having an interior cavity for retaining moisture of a user's eyes; an inner object configured to match and be received in the interior cavity of the outer silicone layer; and a mechanical fastener secured to the inner object for attaching the eye cover to an eye mask; and wherein the eye cover is configured to cover the user's eye and contours of an orbital bone of a user creating a seal around the user's eye.

According to one feature, a portion of the eye cover is made from a silicone material.

According to another feature, the eye cover is made of a malleable material.

According to yet another feature, the inner object is a foam layer.

According to yet another feature, the mechanical fastener is a hook and loop fastener.

According to yet another feature, the eye cover is detachable from the eye mask.

According to one example, an eye cover is provided. The eye cover includes a cavity, located within the outer rim, for retaining moisture of a user's eyes; and a flat surface for affixing a mechanical fastener for detachably securing the eye cover to an eye mask; and wherein the eye cover is configured to cover the user's eye and contours of an orbital bone of a user creating a seal around the user's eye.

According to one feature, the outer rim includes a plurality of.

According to another feature, the mechanical fastener is a hook and loop fastener; and wherein a portion of the eye cover is made from a silicone material.

According to one example, an eye mask is provided. The eye mask includes a body portion configured to cover a user's eyes, the body portion having an interior surface and an opposing exterior surface; a first eye cover, configured to cover a user's eyes, detachably secured to the interior surface of the body portion by a first mechanical fastener; and a second eye cover, configured to cover a user's eyes, detachably secured to the interior surface of the body portion by a second mechanical fastener; and wherein the first eye cover and the second eye cover are each configured to cover the user's eye and contours of an orbital bone of a user creating a seal around the user's eyes.

According to one feature, the body portion comprises a first side strap located at a first end of the body portion; and a second side strap located at a second end of the body portion; and wherein the first and second side straps are secured with a mechanical fastener around the user's head.

According to another feature, each of the first and second mechanical fasteners is a hook and loop fastener.

According to yet another feature, a portion of the first and second eye covers are made from a silicone material.

According to yet another feature, the body portion is made of a malleable material.

According to yet another feature, the first and second eye covers are made of a malleable material.

According to yet another feature, each of the first and second eye covers comprise an outer silicone layer having an interior cavity for retaining moisture of the user's eyes; and an inner object configured to match and be received in the interior cavity of the outer silicone layer.

According to yet another feature, the inner object is a foam layer; and wherein each of the first and second eye covers comprise an outer silicone layer.

According to yet another feature, each of the first and second eye covers further comprise a flat surface for affixing a third mechanical fastener for detachably securing the first and second eye covers to the interior surface of the body portion; and wherein the third mechanical fastener is a hook and loop fastener.

According to yet another feature, each of the first and second eye covers comprise; an outer rim defining a perimeter of the eye cover; and a cavity located within the outer rim.

According to yet another feature, the outer rim has a varying thickness; and wherein the varying thickness gradually decreases from a first side of the outer rim to a second opposing side of the outer rim.

According to yet another feature, the eye mask further comprises one or more relief vents located within an interior section of the outer rim.

### BRIEF DESCRIPTION OF THE DRAWINGS

In the following description, specific details are given to provide a thorough understanding of the embodiments. However, it will be understood by one of ordinary skill in the art that the embodiments may be practiced without these specific details.

FIG. 1 illustrates a front view of an eye mask, according to one aspect of the present disclosure.

FIG. 2 illustrates a back view of the eye mask of FIG. 1.

FIG. 3 illustrates a side view of an eye cover, according to one aspect of the present disclosure.

FIG. 4 illustrates a front exploded view of the eye cover of FIG. 3.

FIG. 5 illustrates a side exploded view of the eye cover of FIG. 3.

FIG. 6 illustrates a top plan view of the eye cover of FIG. 3.

FIG. 7 illustrates a bottom plan view of the eye cover of FIG. 3.

FIG. 8 illustrates a side view of an eye cover, according to another aspect of the present disclosure.

FIG. 9 illustrates a front exploded view of the eye cover of FIG. 8.

FIG. 10 illustrates a side exploded view of the eye cover of FIG. 8.

FIG. 11 illustrates a top plan view of the eye cover of FIG. 8.

FIG. 12 illustrates a bottom plan view of the eye cover of FIG. 8.

FIG. 13 illustrates a top, right perspective view of an eye cover, according to another aspect of the present disclosure.

FIG. 14 illustrates an eye cover on an individual, according to one aspect of the present disclosure.

FIG. 15 a front view of an eye mask, according to one aspect of the present disclosure.

FIG. 16 illustrates a back view of the eye mask of FIG. 15.

FIG. 17 illustrates a side view of the eye mask of FIG. 15.

FIG. 18 illustrates a pair of eye covers on the interior surface of an eye mask positioned to cover the eyes of an individual.

FIG. 19 illustrates a partial view of a pair of eye covers on the interior surface of an eye mask in a first configuration.

FIG. 20 illustrates a partial view of a pair of eye covers on the interior surface of an eye mask in a second configuration.

FIG. 21 illustrates a partial view of an eye mask the pair of eye covers disassembled.

FIG. 22 illustrates a side view of an eye cover of FIG. 16 in a dissembled configuration.

FIG. 23 illustrates a side view of the eye cover of FIG. 22 in an assembled configuration.

### DETAILED DESCRIPTION

The features, nature, and advantages of the present aspects may become more apparent from the detailed description set forth below when taken in conjunction with the drawings in which like reference characters identify correspondingly throughout.

### Overview

Generally speaking, the present disclosure is directed towards an eye mask for treating dry eye and/or irritated eye conditions and which helps control, maintain and/or increase the temperature and humidity around the eyes to aid in limiting evaporation of natural and/or artificial tears. The eye mask includes two detachable eye covers (or eye pieces), each eye cover having a cavity that accommodates the eye or surrounding area. Unlike existing eye masks, the eye mask of the present disclosure may include one or more pressure relief vents to ensure there is no pressure buildup on/around the eye to prevent corneal hypoxia (oxygen deprivation from the cornea).

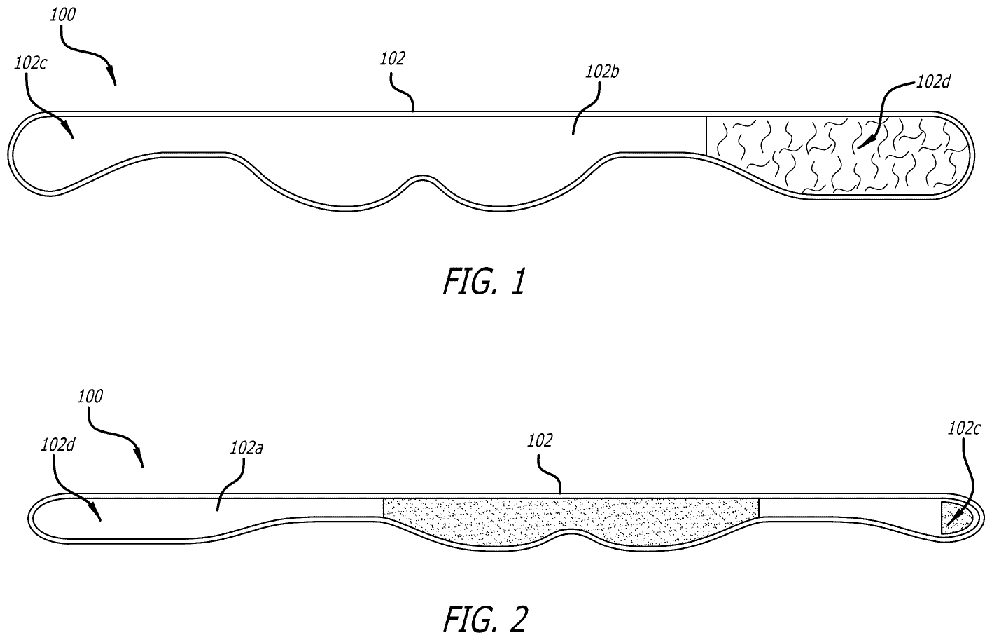

FIGS. 1-2 illustrate front and back views of an eye mask 100 according to one aspect of the present disclosure. The eye mask includes a body portion 102 configured to cover a user's eyes, the body portion 102 having an interior surface 102a and an opposing exterior surface 102b. The body portion 102 further includes a first side strap 102c at a first end of the body portion 102 and a second side strap 102d at a second end of the body portion 102 that can be mutually fixed with a mechanical fastener around the user's head. The mechanical fastener can include, but is not limited to, hook and loop fasteners, magnetic closures, snap buttons, or buckle systems. The first end of the first side strap 102c is opposite the second end of the second side strap 102d.

According to one aspect, the body portion 102 may be made from a malleable material that allows the body portion to be adjusted to conform to a user's unique facial features. However, any material known in the art may be utilized.

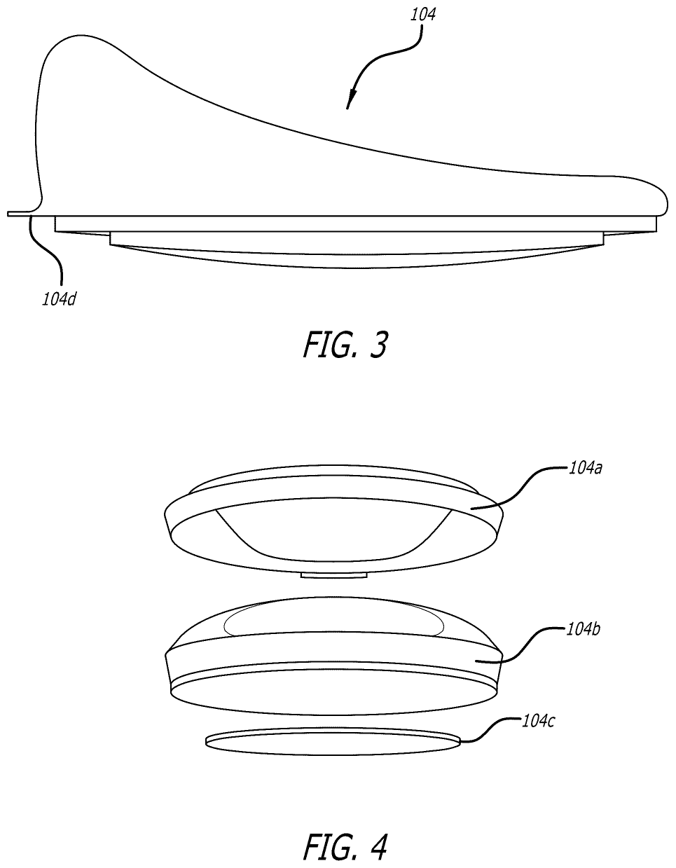

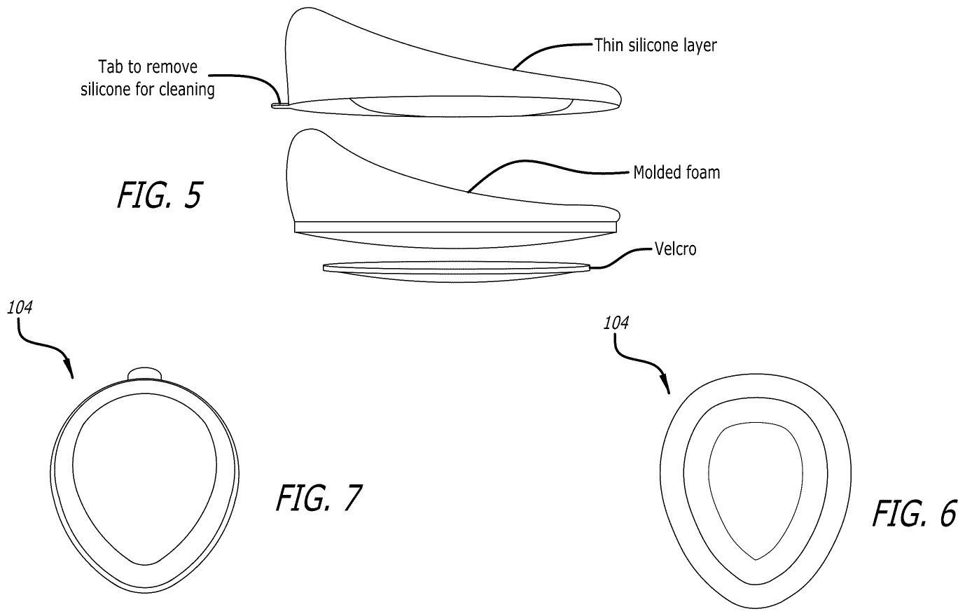

Turning to FIG. 3, an eye cover 104 for detachably securing to the interior surface 102a of the body portion according to one aspect of the present disclosure is illustrated. FIG. 4 illustrates exploded views of the eye cover 104 of FIG. 3. FIG. 5 illustrates a side exploded view of the eye cover of FIG. 3.

According to one example, at least a portion of the eye cover may be made from a silicone material. The eye cover 104 includes an outer silicone layer 104a having an interior cavity configured to receive and accommodate an inner object 104b. In one aspect, the inner object 104b may be a foam layer configured to match the contours of the cavity of the outer silicone layer 104a such that the foam layer fits securely within the cavity. This configuration allows for seamless integration between the outer silicone layer 104a and the molded inner object 104b improving the strength and performance of the eye cover 104. A mechanical fastener 104c may be affixed to the inner object 104b and adapted to be detachably secured to the interior surface 102a of the body portion 102. The eye covers 104 are detachable from the eye mask 100, allowing users to clean them thoroughly before each use ensuring the product's cleanliness and safety, as well as minimizing the risk of irritation or infection caused by accumulated dirt or debris. The mechanical fastener can include, but is not limited to, hook and loop fasteners, magnetic closures, snap buttons, or buckle systems.

According to one aspect, the outer silicone layer 104a includes a tab 104d integrally connected to, and extending vertically outward, from a side of the outer silicone layer 104a providing for easy separation of the outer silicone layer 104a from the molded inner object 104b. Once separated, the components of the eye cover 104 can be easily cleaned. The eye cover 104 may have an almond or oval shape similar to an eye.

Each eye mask 100 can utilize two eye covers 104 and each eye cover 104 attaches to the interior surface 102a of the body portion 102 and can be adjusted in both latitudinal and longitudinal directions. The eye covers 104 can be finely positioned to fit most face sizes and shapes. This adaptability increases the efficacy of creating a complete seal around each eye, ensuring a humid microenvironment, and thus device efficacy, for users with varying facial features and orientations. According to one example, the eye covers 104 are made of a malleable material.

FIG. 6 illustrates a top plan view of the eye cover 104 of FIG. 3. FIG. 7 illustrates a bottom plan view of the eye cover 104 of FIG. 3.

The eye mask 100 of the present disclosure comprises a first eye cover detachably secured to the interior surface 102a of the body portion 102 and a second eye cover detachably secured (or attached) to the interior surface 102a of the body portion 102. Each silicone eye cover has a curved opening that envelops the eyes to ensure a complete moisture seal around the eyes. This creates a humid microenvironment preventing excessive evaporation of tears which minimizes symptoms of dry eye, ultimately improving ocular health. Additionally, by creating a complete seal around the eyes, the silicone eye covers 104 create a barrier against drafts, wind, dust, and dry air which greatly reduces inflammation and dry eye symptom flare ups.

An eye mask has two separate silicone eye covers 104 that attach to the body portion and can be adjusted in latitudinal and/or longitudinal directions allows the eye mask to be finely positioned to fit most face sizes and shapes. This adaptability increases the efficacy of creating a complete seal around each eye, ensuring a humid microenvironment, and thus device efficacy, for users with varying facial features and orientations.

Each eye mask 100 can utilize two eye covers 104 and each eye cover 104 attaches to the interior surface 102a of the body portion 102 and can be adjusted in both latitudinal and longitudinal directions. The eye covers 104 can be finely positioned to fit most face sizes and shapes. This adaptability increases the efficacy of creating a complete seal around each eye, ensuring a humid microenvironment, and thus device efficacy, for users with varying facial features and orientations.

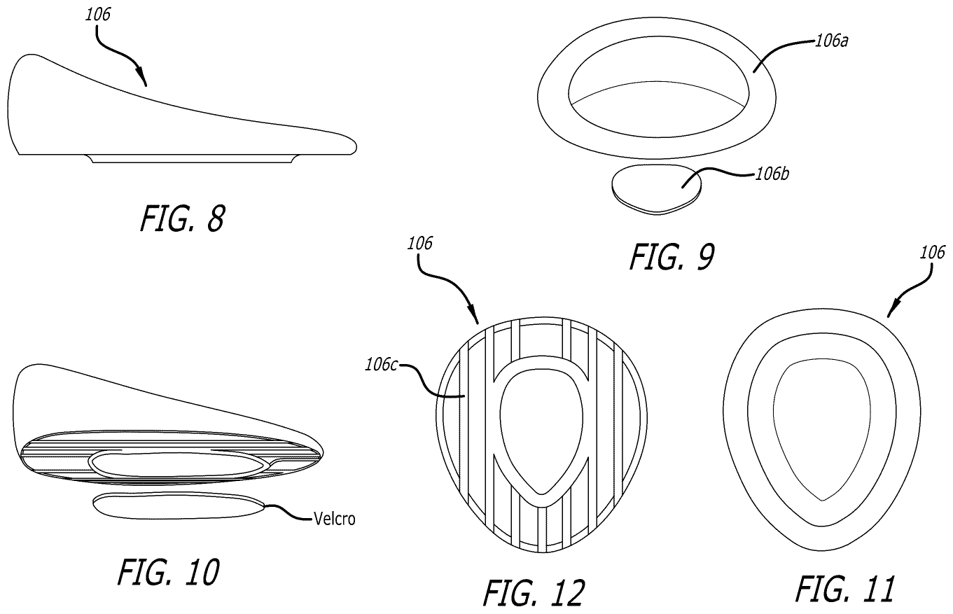

FIG. 8 illustrates a side view of an eye cover 106, according to another aspect of the present disclosure. FIG. 9 illustrates a front exploded view of the eye cover 106 of FIG. 8. FIG. 10 illustrates a side exploded view of the eye cover 106 of FIG. 8.

The eye cover 106 includes an outer smooth layer 106a, providing a sleek and comfortable exterior surface in contact with the skin. Beneath this smooth layer lies a system of ribs 106c, strategically positioned to offer structural support and maintain the shape of the eye cover. These ribs 106c ensure that the eye cover retains its contour, allowing it to cover the user's eye and the contours of an orbital bone of a user creating a seal around the user's eye. Furthermore, by adding ribs 106c, the silicone eye cover can stand up on its own and maintain its structure around the user's orbital bones.

This combination of a smooth outer layer and an underlying ribbed structure provide structural support of the eye cover and may help distribute pressure evenly and enhances the overall comfort and functionality of the eye cover as well as creates a complete seal around each eye to enhance comfort and light blockage. According to one aspect, the bottom of the eye cover 106 includes a flat surface for affixing a mechanical fastener detachably securing the eye cover 106 to the interior surface 102a of the body portion 102. The eye cover 106 may have an almond or oval shape similar to an eye.

The eye covers 106 are detachable from the eye mask 100, allowing users to clean them thoroughly before each use ensuring the product's cleanliness and safety, as well as minimizing the risk of irritation or infection caused by accumulated dirt or debris.

FIG. 11 illustrates a top plan view of the eye cover 106 of FIG. 8. FIG. 12 illustrates a bottom plan view of the eye cover 106 of FIG. 8.

As discussed above, an eye mask includes two separate silicone eye covers that attach to the body portion and can be adjusted in latitudinal and/or longitudinal directions allows the eye mask to be finely positioned to fit most face sizes and shapes. This adaptability increases the efficacy of creating a complete seal around each eye, ensuring a humid microenvironment, and thus device efficacy, for users with varying facial features and orientations.

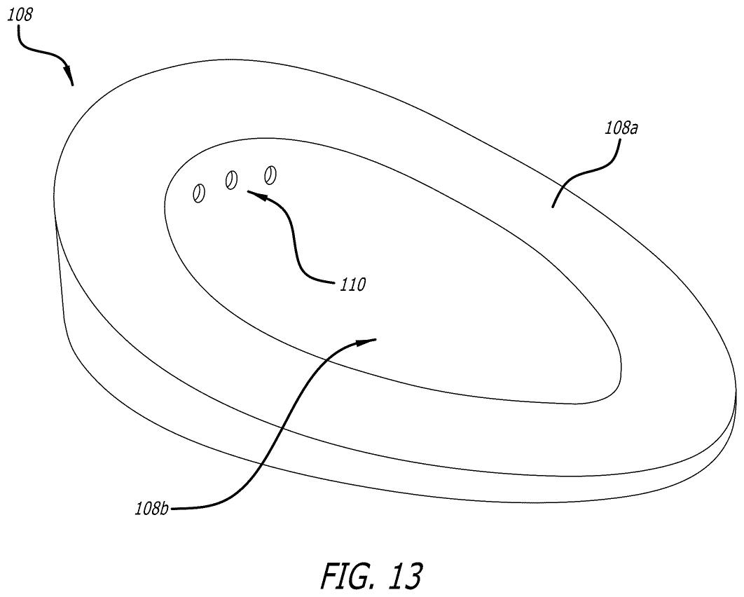

FIG. 13 illustrates a top, right perspective view of an eye cover 108, according to another aspect of the present disclosure. The eye cover 108 may have an almond or oval shape that mimics the natural shape of the eye area of the user ensuring that the eye cover 108 can comfortably fit over the eye. Furthermore, the eye cover 108 may be contoured to conform to the curvature of the eye and the surrounding orbital area for a secure and comfortable fit as well as cover the inner eye area of the individual/user.

As shown in FIG. 13, the eye cover 108 has an outer rim 108a, defining a perimeter of the eye cover 108, and a cavity 108b located within the outer rim 108a. According to one aspect, the outer rim 108a has a varying thickness such that the thickness gradually decreases from a first side of the outer rim 108a to a second opposite side of the outer rim 108a, creating a gradient in thickness along the perimeter. This gradient could serve various purposes, such as optimizing comfort, ensuring even pressure distribution, or accommodating specific design or material properties for enhanced functionality.

According to one aspect, the eye cover 108 may further include one or more relief vents 110 located within an interior section the outer rim 108a for facilitating air flow within the eye cover 108. Although three (3) relief vents 110 are shown, this is by way of example only and the eye cover 108 may include fewer than three (3) relief vents 110 or more than three (3) relief vents 110.

The one or more relief vents 110 prevent pressure buildup in the area surrounding the eye. By allowing air to flow through, these relief vents 110 help to equalize pressure, providing comfort and preventing any negative side effects due to pressure accumulation. Although the one or more relief vents 110 are shown in the in the thickest portion of the outer rim 108a, this is by way of example only and the one or more relief vents 110 there is no pressure buildup on/around the eye.

A mechanical fastener (not shown) may be affixed to a bottom surface of the eye cover 108 and adapted to be detachably secured (or attached) to the interior surface 102a of the body portion 102. The eye covers 108 are detachable from the eye mask 100, allowing users to clean them thoroughly before each use ensuring the product's cleanliness and safety, as well as minimizing the risk of irritation or infection caused by accumulated dirt or debris. The mechanical fastener can include, but is not limited to, hook and loop fasteners, magnetic closures, snap buttons, or buckle systems.

Each eye mask 100 can utilize two eye covers 104 and each eye cover 104 attaches to the interior surface 102a of the body portion 102 and can be adjusted in both latitudinal and longitudinal directions. The eye covers 104 can be finely positioned to fit most face sizes and shapes. This adaptability increases the efficacy of creating a complete seal around each eye, ensuring a humid microenvironment, and thus device efficacy, for users with varying facial features and orientations.

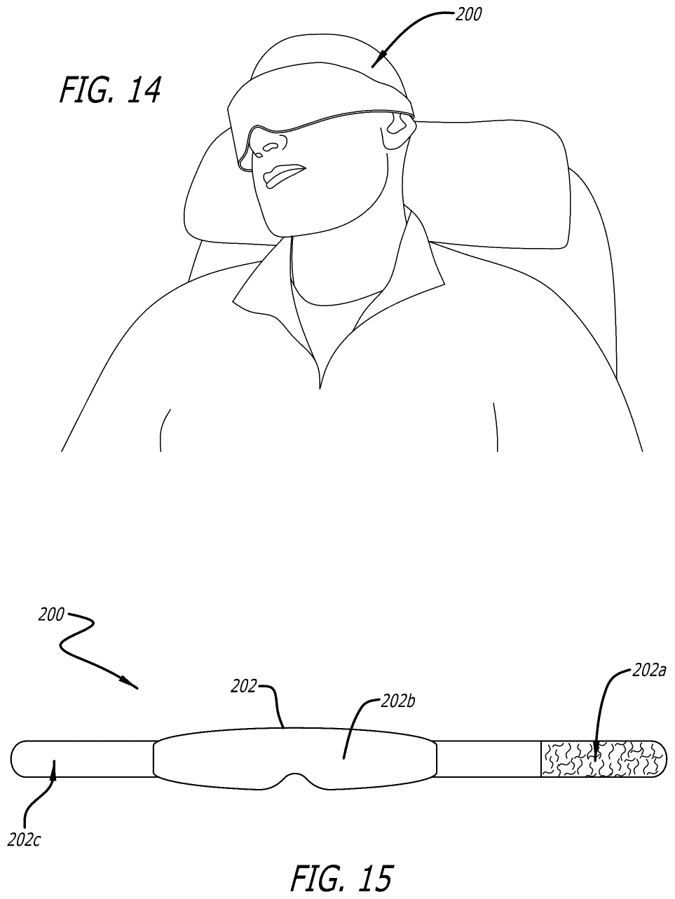

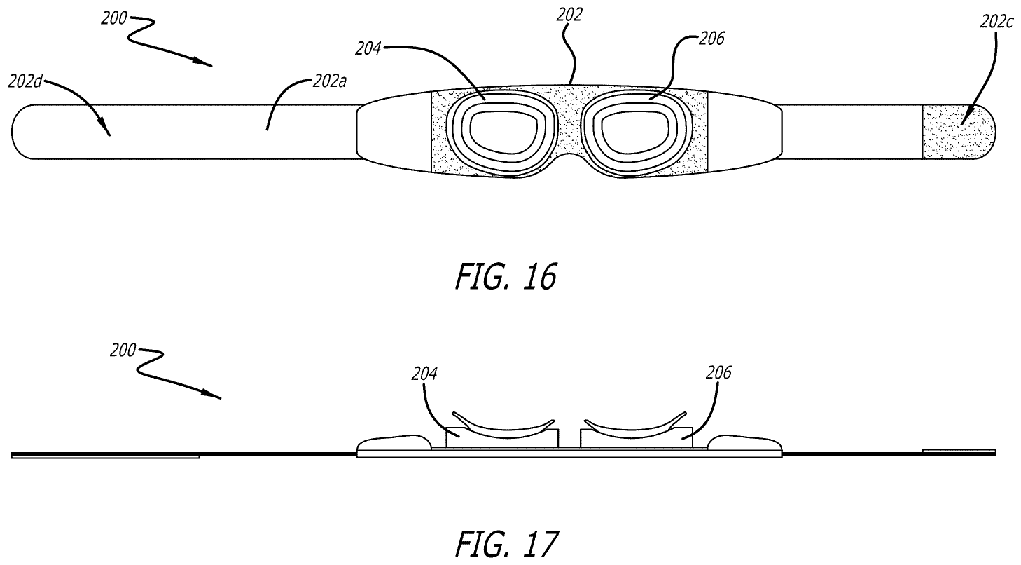

FIG. 14 illustrates an eye mask on an individual, according to one aspect of the present disclosure. FIGS. 15-16 illustrate front and back views of an eye mask 200 according to one aspect of the present disclosure. FIG. 17 illustrates a side view of the eye mask 200 of FIG. 15. The eye mask 200 includes a body portion 202 configured to cover a user's eyes, the body portion 202 having an interior surface 202a and an opposing exterior surface 202b. The body portion 202 further includes a first side strap 202c and a second side strap 202d that can be mutually fixed with a mechanical fastener around the user's head. The mechanical fastener can include, but is not limited to, hook and loop fasteners, magnetic closures, snap buttons, or buckle systems. Additionally, a pair of eye covers 204, 206 are removably attached to the interior surface 202a of the body portion 202 of the eye mask 200.

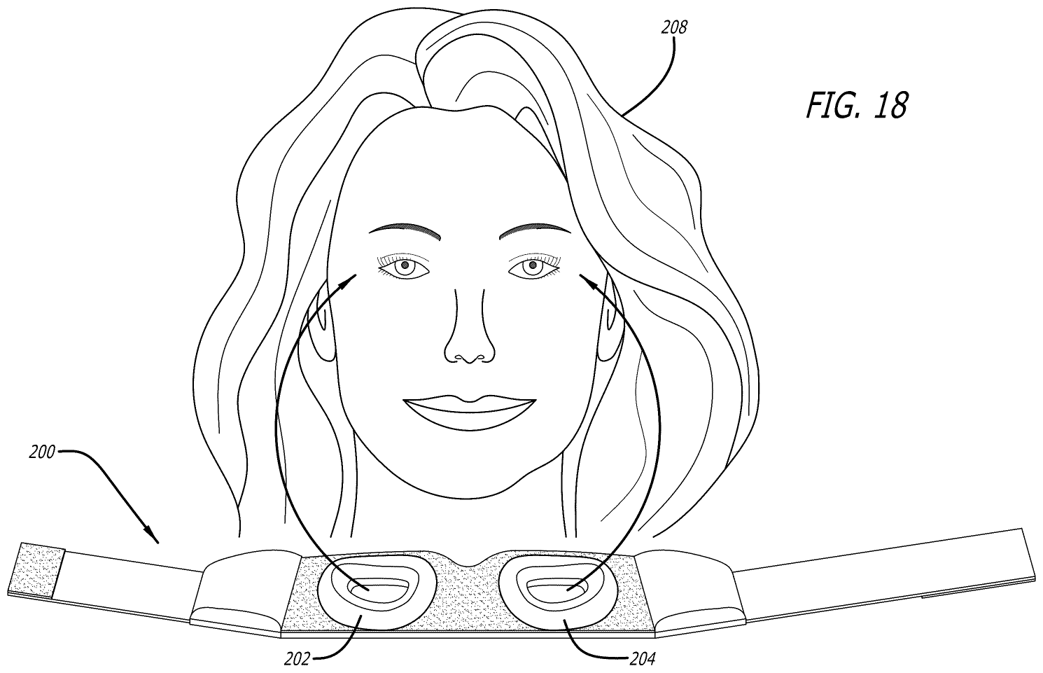

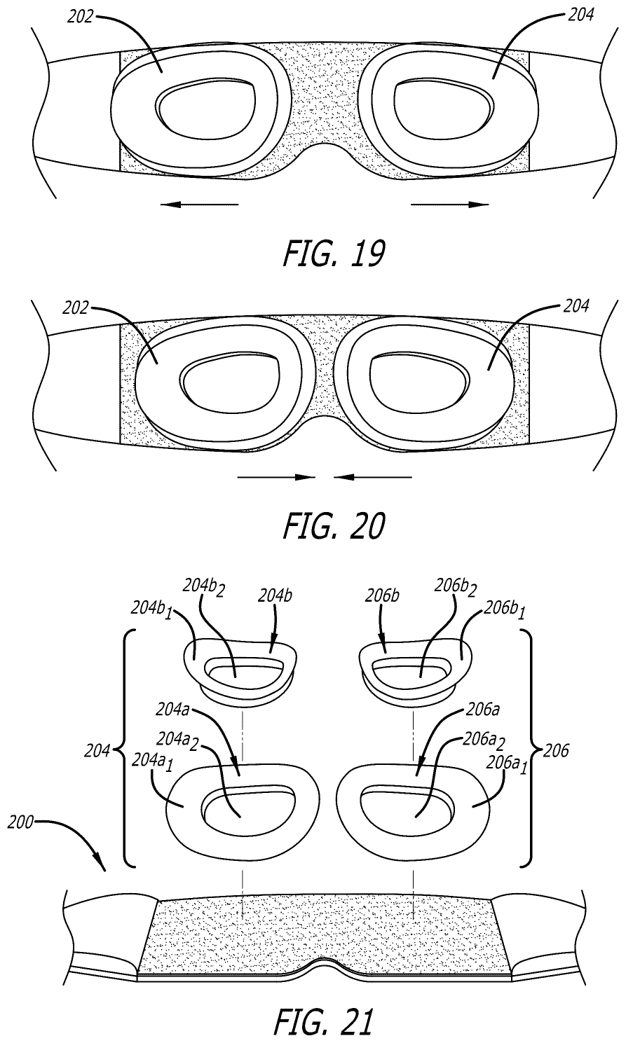

Turning to FIG. 18, the pair of eye covers 204, 206 are illustrated on the interior surface of the eye mask 200 positioned to cover the eyes of an individual 208. The pair of eye covers 204, 206 are removably attached to the interior surface 202a of the body portion 202 of the eye mask 200 and can be adjusted in both the latitudinal and longitudinal directions. FIG. 19 illustrates a partial view of the pair of eye covers 204, 206 on the interior surface 202a of the body portion 202 of the eye mask 200 in a first configuration where the eye covers 204, 206 are separated by a first latitudinal distance. FIG. 20 illustrates a partial view of the pair of eye covers 204, 206 on the interior surface 202a of the body portion 202 of the eye mask 200 in a second configuration where the eye covers 204, 206 are separated by a second latitudinal distance, the first latitudinal distance greater than the second latitudinal distance. As a result, the eye covers can be finely positioned to fit different sized and shaped faces. This adaptability increases the efficacy of creating a complete seal around each eye, ensuring a humid microenvironment, and thus efficacy, for individuals with varying facial features and orientations.

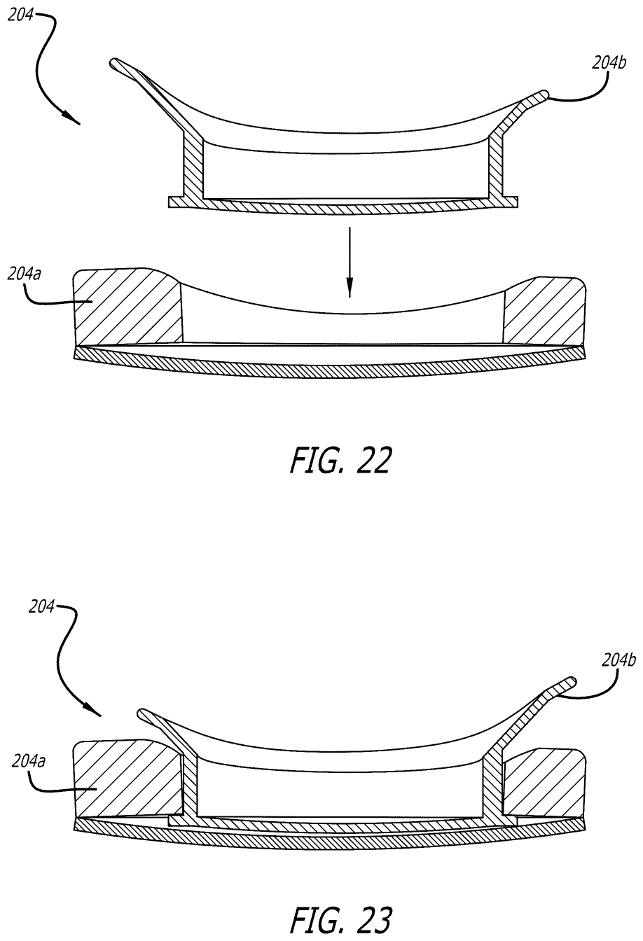

Turning to FIG. 21, a partial view the eye mask 200 showing each eye cover in the pair of eye covers 204, 206 disassembled. FIG. 22 illustrates a side view of an eye cover 204 of FIG. 16 in a dissembled configuration and FIG. 23 illustrates a side view of the eye cover 204 of FIG. 22 in an assembled configuration.

Each of the eye covers 204, 206 includes a support member 204a, 206a and an eyepiece 204b, 206b. According to one example, each of the support members 204a, 206b may have a generally almond or oval shape similar to an eye with an outer rim 204a1 defining a perimeter of the support member 204a, 206a and a cavity 204a2, 206a2 for receiving each eyepiece 204b, 206b. Each eyepiece 204b, 206b may include an outer rim 204b1, 206b1 defining a perimeter of the eyepiece 204b, 206b and a cavity 204b2, 206b2 that accommodates the eye or surrounding area. Alternatively, the support member 204a, 206a may be any other shape known in the art.

In one example, each of the support members 204a, 206a may be made from foam (or foam layer) and covered in fabric, although other materials known in the art may be utilized. In one example, each of the eyepieces 204b, 206b may be made from a silicone material. Optionally, each of the eyepieces 204b, 206b may attach to a piece of fabric and foam (or foam layer) to support user comfort.

As shown, the eye covers 204, 206 are detachable from the interior surface 202a of the body portion 202 of the eye mask 200 to allow for a thorough cleaning before use. This ensures the cleanliness and safety of the eye covers 204, 206, as well as the body portion 202, of the eye mask 200 minimizing the risk of irritation or infection caused by accumulated dirt or debris.

According to one aspect, the body portion 202 may be made from a malleable material that allows the body portion to be adjusted to conform to a user's unique facial features. However, any material known in the art may be utilized.

“Comprise” and variations, such as “comprising” and “comprises,” are not intended to exclude other additives, components, integers, or steps. “A”, “an,” and “the” and similar referents used herein are to be construed to cover both the singular and the plural unless their usage in context indicates otherwise. The word “exemplary” is used herein to mean “serving as an example, instance, or illustration.” Any implementation or embodiment described herein as “exemplary” is not necessarily to be construed as preferred or advantageous over other embodiments or implementations. Likewise, “embodiments” does not require that all embodiments include the discussed feature, advantage or mode of operation.

“Aspects” do not require that all aspects of the disclosure include the discussed features, advantages, or modes of operation. “Coupled” is used herein to means the direct or indirect coupling between two objects. For example, if object A physically touches or couples to object B, and object B touches or couples to object C, then objects A and C may still be considered coupled to one another, even if they do not directly physically touch each other.

One or more of the components and functions illustrated in the FIGS. may be rearranged and/or combined into a single component or embodied in several components without departing from the invention. Additional elements or components may also be added without departing from the invention. Additionally, the features described herein may be implemented in software, hardware, as a business method, and/or combination thereof.

While certain exemplary embodiments have been described and shown in the accompanying drawings, it is to be understood that such embodiments are merely illustrative of and not restrictive on the broad invention, and that this invention is not be limited to the specific constructions and arrangements shown and described, since various other modifications may occur to those ordinarily skilled in the art.

1. An eye cover, comprising:
an outer silicone layer having an interior cavity for retaining moisture of a user's eye;
an inner object configured to match and be received in the interior cavity of the outer silicone layer; and
a mechanical fastener secured to the inner object for attaching the eye cover to an eye mask; and
wherein the eye cover is configured to cover the user's eye and contours of an orbital bone of a user creating a seal around the user's eye.

2. The eye cover of claim 1, wherein a portion of the eye cover is made from a silicone material.

3. The eye cover of claim 1, wherein the eye cover is made of a malleable material and the inner object is a foam layer.

4. The eye cover of claim 1, wherein the mechanical fastener is a hook and loop fastener.

5. The eye cover of claim 1, wherein the eye cover is detachable from the eye mask.

6. The eye cover of claim 1, further comprising:
an outer rim defining a perimeter of the eye cover;
a cavity located within the outer rim; and
one or more relief vents located within an interior section of the outer rim.

7. An eye cover, comprising:
an outer rim defining a perimeter of the eye cover;
a cavity, located within the outer rim, for retaining moisture of a user's eyes; and
a flat surface for affixing a mechanical fastener for detachably securing the eye cover to an eye mask; and
wherein the eye cover is configured to cover the user's eye and contours of an orbital bone of a user creating a seal around the user's eye.

8. The eye cover of claim 7, wherein the outer rim includes a plurality of ribs.

9. The eye cover of claim 7, wherein the mechanical fastener is a hook and loop fastener; and wherein the eye cover is made from a silicone material.

10. An eye mask, comprising:
a body portion configured to cover a user's eyes, the body portion having an interior surface and an opposing exterior surface;
a first eye cover, configured to cover a user's eyes, detachably secured to the interior surface of the body portion by a first mechanical fastener; and
a second eye cover, configured to cover a user's eyes, detachably secured to the interior surface of the body portion by a second mechanical fastener; and
wherein the first eye cover and the second eye cover are each configured to cover the user's eye and contours of an orbital bone of a user creating a seal around the user's eyes.

11. The eye mask of claim 10, wherein the body portion comprises:
a first side strap located at a first end of the body portion; and
a second side strap located at a second end of the body portion; and
wherein the first and second side straps are secured with a mechanical fastener around a user's head.

12. The eye mask of claim 10, wherein each of the first and second mechanical fasteners is a hook and loop fastener.

13. The eye mask of claim 10, wherein a portion of the first and second eye covers are made from a silicone material.

14. The eye mask of claim 10, wherein the body portion is made of a malleable material.

15. The eye mask of claim 10, wherein the first and second eye covers are made of a malleable material.

16. The eye mask of claim 10, wherein each of the first and second eye covers comprise:
an outer silicone layer having an interior cavity for retaining moisture of the user's eyes; and
an inner object configured to match and be received in the interior cavity of the outer silicone layer.

17. The eye mask of claim 16, wherein the inner object is a foam layer; and wherein each of the first and second eye covers comprise an outer silicone layer.

18. The eye mask of claim 17, wherein each of the first and second eye covers further comprise a flat surface for affixing a third mechanical fastener for detachably securing the first and second eye covers to the interior surface of the body portion; and wherein the third mechanical fastener is a hook and loop fastener.

19. The eye mask of claim 10, wherein each of the first and second eye covers comprise:
an outer rim defining a perimeter of the eye cover; and
a cavity located within the outer rim.

20. The eye mask of claim 19, wherein the outer rim has a varying thickness; and wherein a varying thickness gradually decreases from a first side of the outer rim to a second opposing side of the outer rim.
